(19) United States
(12) Plant Patent Application Publication
(10) Pub. No.: US 2026/0130301 P1
(43) Pub. Date: May 7, 2026

(54) SWEET ORANGE PLANT NAMED 'OLL-DC-3-36'

(50) ;

(71) Applicant: Florida Foundation Seed Producers, Inc., Marianna, FL (US)

(72) Inventor: Jude GROSSER, Winter Haven, FL (US)

(21) Appl. No.: 19/381,461

(22) Filed: Nov. 6, 2025

Related U.S. Application Data
(60) Provisional application No. 63/717,552, filed on Nov. 7, 2024.

(51) Int. Cl. A01H 6/78; A01H 5/02; A01H 5/08

(52) U.S. Cl. CPC A01H 6/785; A01H 5/02; A01H 5/08
USPC PLT/202

(57) ABSTRACT

A new and distinct cultivar of sweet orange plant named ‘OLL-DC-3-36’ selected for processing and fresh market, characterized by an increased resistance to HLB. Fruit of OLL-DC-3-36′ are round.

### CROSS-REFERENCE TO RELATED APPLICATIONS

This application claims the benefit of U.S. Provisional Patent Application No. 63/717,552, filed Nov. 7, 2024, entitled SWEET ORANGE PLANT NAMED ‘OLL-DC-3-36’, which is incorporated by reference herein in its entirety.

### ACKNOWLEDGEMENT OF FEDERAL RESEARCH SUPPORT

N/A.

Genus and species:

Citrus sinensis.

Cultivar denomination:

‘OLL-DC-3-36’.

### BACKGROUND OF THE NEW CULTIVAR

The present invention relates to a new and distinct cultivar of sweet orange, botanically known as Citrus sinensis, and hereinafter referred to by the name ‘OLL-DC-3-36’. Citrus sinensis is a member of Rutaceae.

The new sweet orange cultivar ‘OLL-DC-3-36’ is a nucellar seedling selected from a population of over 214 nucellar seedlings grown from seed derived from previously released sweet orange cultivar ‘OLL-8’ (U.S. Plant Pat. No. 26,087), from an OLL (Orie and Louise Lee) series of sweet oranges. The mother plant ‘OLL-8’ is a somaclone regenerated from embryogenic callus (tissue culture) of the original unstable OLL sweet orange, with high quality fruit that usually matures in the standard ‘Valencia’ time period (not patented). The ‘OLL-8’ nucellar seedling population was planted in Dade City, Florida, in April of 2012, and all trees were planted on their own roots (no grafting). After four years in the ground, the commercial grove was devastated by huanglongbing (HLB), or citrus greening, and the grove was sold for real estate development. Thus, in spring of 2016, the entire population was assessed for HLB, and ‘OLL-DC-3-36’ was one of only two clones that were identified as having no HLB symptoms. Trees were still completely juvenile at this time, showing typical upright growth and thorniness, and the trees were not yet fruiting. Both selected trees tested negative for CLas using qPCR (the only two clones to do so), but this was unexpected because the entire population of trees were vigorously growing trees with lots of new vegetative flush to attract psyllids, and there was plenty of CLas inoculum in the adjacent collapsing commercial blocks. This suggested some higher level of HLB tolerance or possibly resistance. ‘Valencia’ is considered to be the “gold standard” of processing oranges worldwide; however, OLL oranges produce juice with better color and almost always better flavor.

### SUMMARY OF THE INVENTION

The new sweet orange cultivar ‘OLL-DC-3-36’ has not been observed under all possible environmental conditions. The phenotype of the new cultivar may vary with variations in environment and cultural practices such as temperature, light intensity, fertilization, irrigation, and application of plant growth regulators without any change in genotype.

The new sweet orange cultivar ‘OLL-DC-3-36’ produces round oranges with internal and external color similar to that of ‘Rhode Red Valencia’ (not patented). This cultivar is selected for processing and fresh market and has increased HLB tolerance. True-to-typeness of ‘OLL-DC-3-36’ was provided by reset trees on super-root mutant ‘UFR-1’ (U.S. Plant Pat. No. 27,277) clone #28 and ‘UFR-1’ clone #55 rootstocks, with 3.5-year-old trees (HLB+) planted in St. Cloud, Florida, making up to 11.9 brix 6-8 weeks prior to expected harvest date (Table 1). This is important, as there has been a brix crisis in both ‘Hamlin’ (not patented) and ‘Valencia’ (not patented) juice statewide in Florida the past two seasons, with juices from both oranges averaging less brix than what is needed for Grade A standard NFC juice. Combined with the right rootstock, young trees of ‘OLL-DC-3-36’ are capable of making Grade A level juice. ‘OLL-DC-3-36’ trees grow faster and larger than trees of ‘Valencia’ (Table 2). For the 2024harvest, fruit of both ‘OLL-DC-3-36’ and ‘OLL-DC-3-40’ passed maturity standards in mid-February, whereas standard ‘Valencia’ did not (Table 3).

There are no data supporting long-term field performance or yield of the new cultivar ‘OLL-DC-3-36’, although numerous young trees observed growing on ‘US-942’ (not patented) rootstock have set very good crops the past and current season. No obvious tree-debilitating disease problems have been observed in the trees or fruit currently grown, but systematic resistance testing has not been yet explored. Trees of the new cultivar ‘OLL-DC-3-36’ are susceptible to citrus canker, similar to the mother clone ‘OLL-8’. Citrus canker lesions have been found on leaves and a few fruit of ‘OLL-DC-3-36’, suggesting that its canker tolerance is less than that of ‘Valencia’, and probably more similar to that of ‘Hamlin’. Because of its potentially enhanced HLB tolerance, ‘OLL-DC-3-36’ is suited for widespread planting in Florida.

The new cultivar ‘OLL-DC-3-36’ can be compared to the commercial cultivars ‘OLL-8’ and Valencia ‘SPB-1-14-19’, but is showing better HLB tolerance. OLL clones have higher brix and color scores than standard Valencia, as well as better HLB tolerance.

### DESCRIPTION OF THE FIGURES

This new sweet orange cultivar ‘OLL-DC-3-36’ is illustrated by the accompanying photographs which show the tree's from, foliage and fruit. The colors shown are as true as can be reasonably obtained by conventional photographic procedures. The photographs are of a tree approximately 4 years old.

FIG. 1 shows a close-up view of the mature fruit from the new cultivar ‘OLL-DC-3-36’ with the rind present and cross-sectional view of the fruit when cut in the center (photo taken April 2024);

FIG. 2 shows overall tree growth habit of the new cultivar ‘OLL-DC-3-36’;

FIG. 3 shows a cluster with flower buds of the new cultivar ‘OLL-DC-3-36’ in different growth stages;

FIG. 4 shows a cluster with flowers of the new cultivar ‘OLL-DC-3-36’;

FIG. 5 shows fruit on tree of the new cultivar ‘OLL-DC-3-36’;

FIG. 6 shows trunk and branches of a tree of the new cultivar ‘OLL-DC-3-36’;

FIG. 7 shows fully expanded leaves of the new cultivar ‘OLL-DC-3-36’ with a ruler for size estimates;

FIG. 8 shows different growth stages of flowers of the new cultivar ‘OLL-DC-3-36’ with a ruler for size estimates;

FIG. 9 shows flower diameter and flower depth of flowers of the new cultivar ‘OLL-DC-3-36’ with a ruler for size estimates;

FIG. 10 shows a close-up view of a fully expanded flower of the new cultivar ‘OLL-DC-3-36’ with a ruler for size estimates;

FIG. 11 shows seed of the new cultivar ‘OLL-DC-3-36’ with a ruler for size estimates;

FIG. 12 shows a comparison of trees of the new cultivar ‘OLL-DC-3-35’ (left) with trees of ‘Valencia’ (right); and

FIG. 13 shows field trees of the new cultivar ‘OLL-DC-3-36’ showing HLB tolerance.

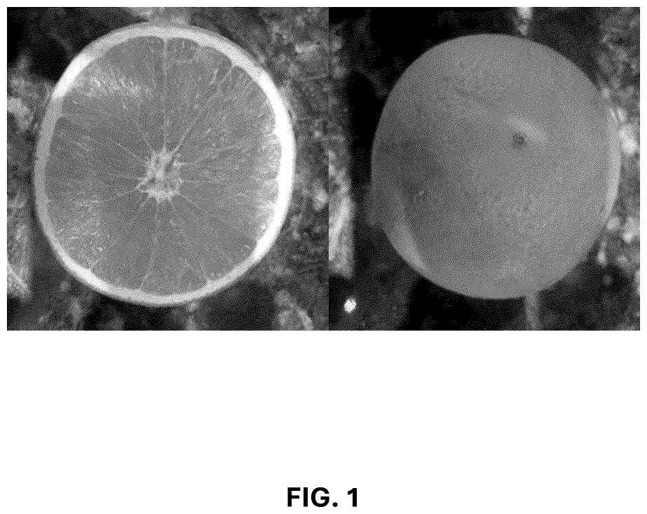

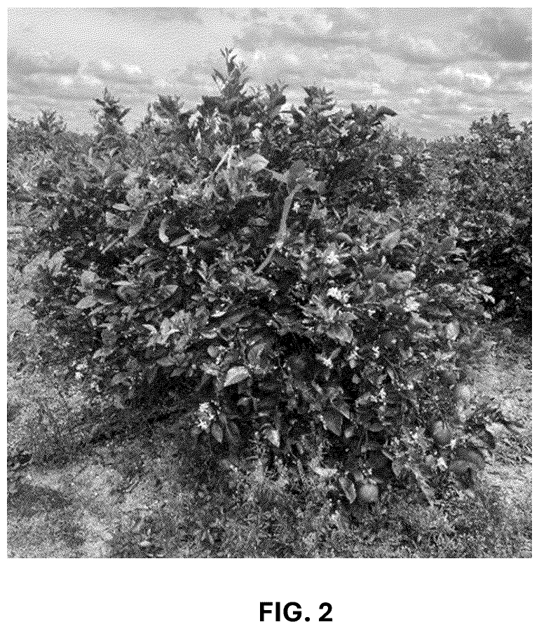

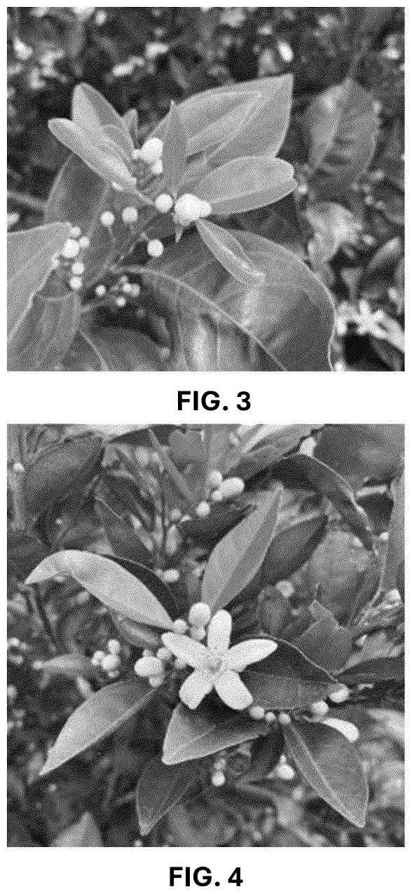

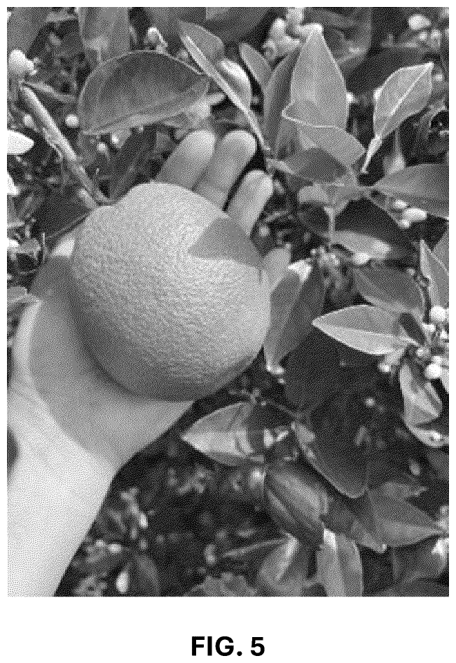

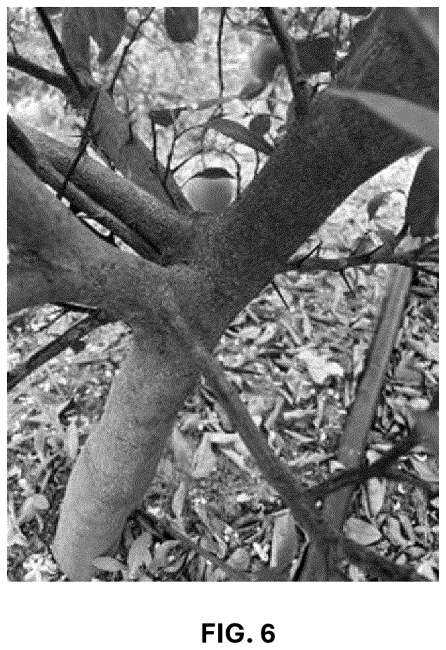

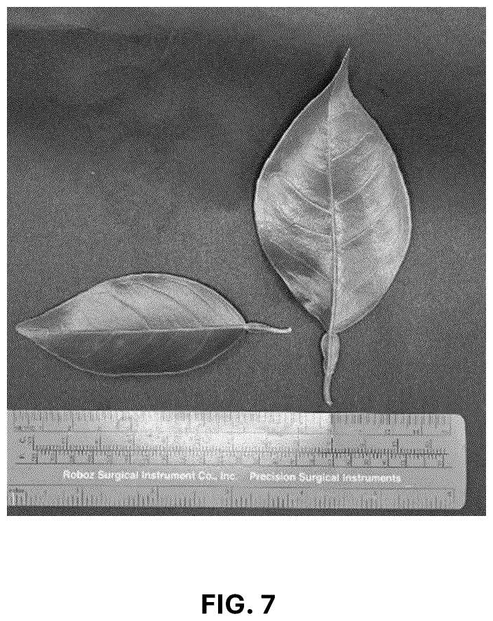

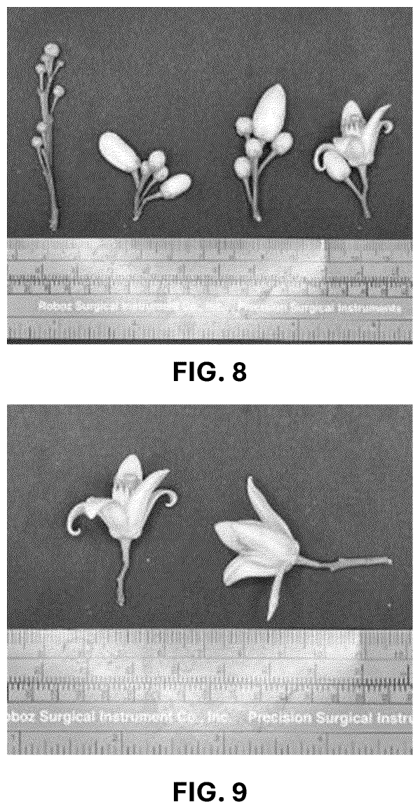

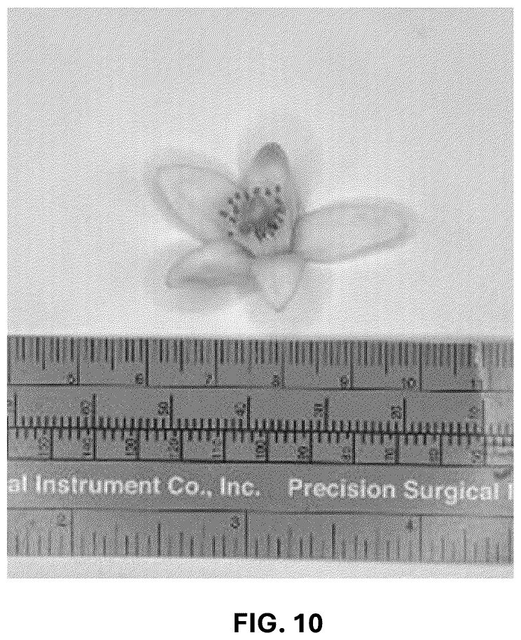

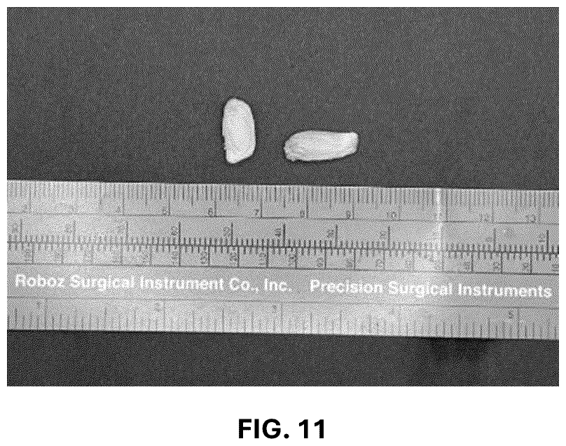

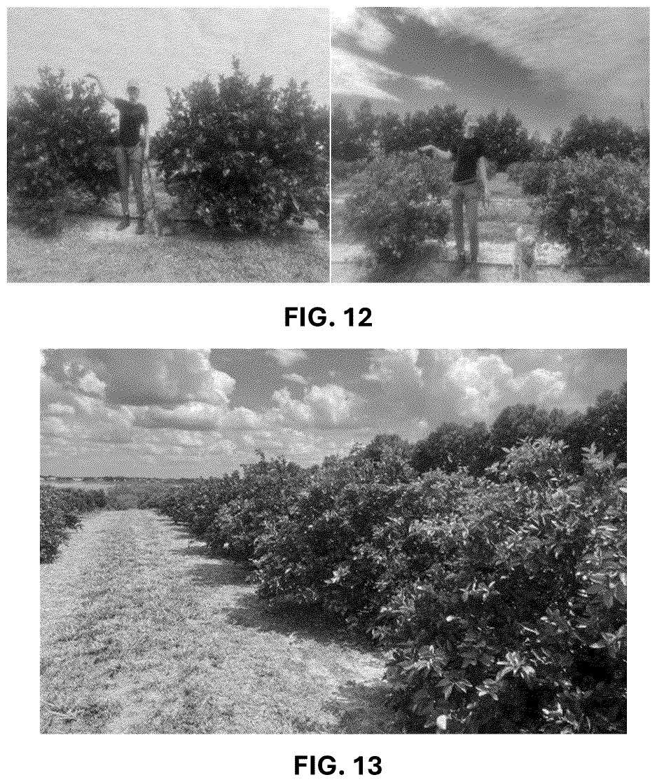

### DETAILED BOTANICAL DESCRIPTION OF THE CULTIVAR

The present botanical description is that of the new cultivar ‘OLL-DC-3-36’ grown on 4.5-year-old trees growing on ‘US-942’ rootstock (C. reticulata x Poncirus trifoliata) in Lake Alfred, Florida. The colors (except those in common terms) are described from R.H.S. Colour Chart published by the Royal Horticultural Society in London (2007, 5^{th} edition), in association with the Flower Council of Holland.

### Botanical Description

Botanical classification:
   Family.—Rutaceae.
   Botanical name.—Citrus sinensis.
   Common name.—Sweet orange.
   Cultivar.—‘OLL-DC-3-36’.
Parentage: OLL (Orie & Louise Lee) Sweet Orange #8 (‘OLL-8’).
Tree:
   Ploidy.—Diploid.
   Size.—Medium.
   Width across the row.—2.81 meters to 3 meters on average.
   Width down row.—2.78 meters to 3 meters on average.
   Tree height.—1.91 meters on average.
   Tree canopy diameter.—7.92 meters on average.
   Vigor.—High.
   Density.—Dense.
   Tree shape (form).—The shape of the tree is obloid with upright and lateral branches growing, but tending toward medium angles after fruit has commenced.
   Growth habit (current season).—Drooping.
Trunk:
   Trunk diameter.—23.3 cm on average at 30 cm above the ground.
   Trunk texture.—Smooth.
   Trunk bark color.—R.H.S. 199A (Grey-Brown Group).
Branches:
   Branch length.—1.50 meters on average.
   Branch diameter.—9 cm on average.
   Crotch angle.—On average, the angle is less than 90 degrees in the middle of the tree and some crotch angles on the outside are more than 90 degrees.
   Branch texture.—Less rough than the trunk with small thorns.
   Branch color.—RHS 199C (Grey-Brown Group).
Leaves:
Size (lamina average):
   Length.—102 mm on average.
   Width.—69 mm on average.
   L/W ratio.—1.48 on average.
   Thickness.—Relatively thick (approximately 0.25 mm).
   Type.—Simple (unifoliate).
   Shape.—Elliptical.
   Apex.—Slightly retuse.
   Base.—Acute.
   Margin.—Entire, slightly undulate.
Surface:
   Upper surface.—Smooth.
   Lower surface.—Smooth with medium veins that are pinnately netted.
Color:
   Upper surface (adaxial).—RHS 137 B (Yellow-Green Group).
   Lower surface (abaxial).—RHS 146 B (Yellow-Green Group).
Petiole:
   Shape and attachment.—Brevipetiolate. The junction between petiole and lamina is articulate.
   Shape (petiole wing).—Obovate with narrow end at the base.
   Length.—25 mm on average.
   Width.—5 mm on average.
   Color.—RHS 144 A (Yellow-Green Group).
Flowers:
   Type.—Hermaphrodite.
   Flowers bearing.—Single or cluster, with each cluster having 5 to 6 flowers. Some flowers can grow individually.
   Flower diameter.—26.6 mm on average.
   Flower depth.—18.8 mm to 20 mm on average.
   First bloom.—Observed around mid to late February.
   Full bloom.—March.
   Fragrance.—Fragrant.
Flower bud size:
   Length.—1.6 mm on average for the initially visible flower bud; 12.4 mm on average in the mature bud.
   Diameter.—1.3 mm on average in the initial visible flower bud; 8.5 mm on average in the matured bud.
   Flower bud shape.—Initially with a round shape and, when totally mature, as an elongated olive shape.
   Flower bud color (RHS).—RHS 144C (Yellow-Green Group) for the initial visible flower bud; RHS 157B (Green-White Group) for mature buds.
   Fertility.—Appears to be self-fertile.
Petals:
   Number.—5.
   Petal length.—18.7 mm on average.
   Petal width.—6.6 mm on average.
   Petal shape.—Recurved/cruciform.
   Apex shape.—Smooth with an obtuse angle.
   Base shape.—Obtuse
Petal color:
   Upper Surface.—RHS 155A (White Group).
   Lower surface.—RHS 155B (White Group).
   Margin.—Smooth.
Sepal:
   Number (per flower).—3-5 per flower.
   Shape.—Recurved/cruciform.
   Length.—3.1 mm on average.
   Width.—4.2 mm on average.
   Apex shape.—Acute.
   Margin.—Smooth.
Sepal color:
   Upper surface.—RHS 142 C (Green Group).
   Lower surface.—RHS 142 D (Green Group).
Pedicel:
   Length.—7.7 mm on average.
   Diameter.—1.1 mm on average.
   Color.—RHS 144 C (Yellow-Green Group).
Stamen:
   Number.—23 per flower on average.
   Length.—12.1 mm on average.
Anther:
   Length.—1.9 mm on average.
   Width.—0.8 mm on average.
   Color.—RHS 14A (Yellow Orange Group).
   Pollen color (general).—RHS 163 A (Greyed-Yellow Group).
Pistil:
   Number.—1.
   Length.—12.3 mm on average.
   Color.—RHS 145 C (Yellow-Green Group).
   Style length.—7 mm on average.
   Style diameter.—1.7 mm on average.
   Style color RHS.—RHS 150 B (Yellow-Green Group).
   Ovary shape.—Oval.
   Ovary diameter.—3 mm on average.
   Ovary color.—RHS 144 A (Yellow Green Group).
Fruit:
   Fruit weight.—165 on average/fruit.
Size: Uniform.
   Diameter.—65 mm on average.
   Length.—67 mm on average.
   Shape.—Round and earth-shaped.
   Shape (cross-section).—Round.
   Apex.—Truncated with shallow dent mark.
   Base.—No neck, with slightly rough peel surface.
Rind:
   Adherence: Albedo (mesocarp) to fresh (endocarp) is strong and relatively difficult to separate.
   Thickness: Approximately 4 mm, thin to medium compared to other commercial sweet orange varieties.
   Texture: Firm.
Color:
   Flavedo (epicarp).—RHS N25B (Orange Group).
   Albedo (mesocarp).—RHS 18D (Yellow-Orange Group).
   Endocarp.—RHS N25C (Orange Group).
   Stylar end.—Closed.
   Oil cell density.—Approximately 38 per mm².
Flesh:
   Number of segments.—10 to 12 segments per fruit on average.
   Segment walls.—Soft, but of sufficient strength to maintain integrity as separated.
   Juice.—Abundant.
   Color.—Uniformly colored, RHS 25B (dark orange) (up to 40 color score via State House testing).
   Texture.—Soft.
Vesicles: Medium thickness.
   Length.—10 mm on average.
   Width.—2.5 mm on average.
Eating quality:
   Sucrose content (average Brix).—9-11.92 (in young trees, approximately 3-5 years of age, with HLB).
   Acidity.—0.91 on average.
   Sugar:acid ratio.—13.1 (in young trees with HLB; higher brix and ratio are expected if harvested later or from older trees).
Seeds:
   Type.—Polyembryonic.
   Number.—Between 3 to 4 seeds (3.4 on average).
   Shape.—Variable, typically clavate/club shaped to deltoid shaped.
   Size.—
   Length.—10 mm to 16 mm on average.
   Width.—5 mm to 7 mm on average.
Seed coat color:
   Outer Surface.—RHS 157A (Green-White Group) and slightly wrinkled.
   Inner surface.—RHS 165C (Greyed-Orange Group).
   Cotyledon color.—RHSb155A (White-Group).

### Data:

TABLE 1
Juice quality data from 3.5-year-old reset trees of ‘OLL-DC-3-36’,
as well as other OLL series clones, from oranges harvested in
January 2023 from St. Cloud, Florida (6-8 weeks prior to expected full maturity).

| | | Wt. | Wt. | Wt. Juice | | | | Lbs. | |
|---|---|---|---|---|---|---|---|---|---|
| Sample | | Sample | Juice | per Box | % | Total | | Solids | Juice |
| ID | Scion/rootstock | (lbs.) | (lbs.) | (lbs.) | Acid | ° Brix | Ratio | per Box | Color |
| RIT9 | ‘OLL-DC-3-40’/‘UF-1’ | 4.54 | 2.58 | 51.145 | 1.50 | 11.50 | 7.67 | 5.88 | 36.8 |
| | CL.55 ‘Super Roots’ | | | | | | | | |
| RIT11 | ‘OLL-3-IR’ 46x ‘20-04- | 11.07 | 6.41 | 52.114 | 1.31 | 10.26 | 7.83 | 5.35 | 36.2 |
| | 42’ | | | | | | | | |
| R1T12 | ‘OLL-DC-3-36’/‘UF-1’ | 4.96 | 2.97 | 53.891 | 1.15 | 11.30 | 9.83 | 6.09 | 37.1 |
| | CL.55 ‘Super Roots’ | | | | | | | | |
| RIT13 | ‘OLL-DC-3-40’/‘UF-1’ | 2.99 | 1.57 | 47.258 | 1.25 | 10.40 | 8.32 | 4.92 | 35.5 |
| | CL.28 ‘Fast Eddy’ | | | | | | | | |
| R2T6 | ‘OLL-DC-3-40’/‘UF-1’ | 17.20 | 9.97 | 52.169 | 1.06 | 10.06 | 9.49 | 5.25 | 37.3 |
| | CL.2 ‘Super Roots-2’ | | | | | | | | |
| R2T14 | ‘OLL-DC-3-40’/‘UF-1’ | 5.81 | 3.41 | 52.823 | 0.97 | 10.10 | 10.41 | 5.33 | 36.7 |
| | CL.28 ‘Fast Eddy’ | | | | | | | | |
| R2T15 | ‘OLL-DC-3-36’/‘UF-1’ | 4.81 | 2.80 | 52.391 | 1.17 | 10.70 | 9.15 | 5.61 | 36.1 |
| | CL.28 ‘Fast Eddy’ | | | | | | | | |
| R2T16 | ‘OLL-DC-3-36’/‘UF-1’ | 12.40 | 7.35 | 53.347 | 1.38 | 11.92 | 8.64 | 6.36 | 36.3 |
| | CL.55 ‘Super Roots’ | | | | | | | | |
| R4T1 | ‘OLL-DC-3-36’/‘UF-1’ | 13.22 | 7.59 | 51.672 | 1.04 | 10.20 | 9.81 | 5.27 | 36.7 |
| | CL.2 ‘Super Roots-2’ | | | | | | | | |
| R7T28 | ‘OLL-DC-3-36’/‘UF-1’ | 18.28 | 10.55 | 51.942 | 1.24 | 10.72 | 8.65 | 5.57 | 36.3 |
| | CL.2 ‘Super Roots-2’ | | | | | | | | |

Trees were HLB+ and grown with no psyllid control.

TABLE 2
Comparison of 4-year-old trees of ‘OLL-DC-3-36’ grown on ‘US-942’ rootstock
(‘OLL-DC-3-36’/‘US-942’), ‘OLL-DC-3-40’ grown on ‘US-942’
(‘OLL-DC-3-40’/‘US-942’), and ‘Valencia’ grown on ‘US-942’ rootstock (‘Valencia’/‘US-942’)
for tree size and yield, average of 10 trees per selection (data collected February 2024).

| | | | Tree | | | | Tree Canopy | | | | Boxes/ |
|---|---|---|---|---|---|---|---|---|---|---|---|
| Scion/ | | | Height (m) | Scion/ | | | Volume (m) | Scion/ | | | Tree |
| Rootstock | | | Mean* | Rootstock | | | Mean* | Rootstock | | | Mean* |
| ‘OLL-DC-3-40’/ | A | | 2.0 | ‘OLL-DC-3-40’/ | A | | 9.3 | ‘OLL-DC-3-36’/ | A | | 0.4 |
| ‘US-942’ | | | | ‘US-942’ | | | | ‘US-942’ | | | |
| ‘OLL-DC-3-36’/ | A | | 1.9 | ‘OLL-DC-3-36’/ | A | | 7.9 | ‘OLL-DC-3-40’/ | | B | 0.1 |
| ‘US-942’ | | | | ‘US-942’ | | | | ‘US-942’ | | | |
| ‘Valencia’/ | | B | 1.4 | ‘Valencia’/ | | B | 5.4 | ‘Valencia’/ | | B | 0.1 |
| ‘US-942’ | | | | ‘US-942’ | | | | ‘US-942’ | | | |

*Means not connected by the same letter are significantly different (x = 0.05).

TABLE 3
Pilot Plant Juice Quality Data from OLL clones, including ‘OLL-DC-3-36’ and
‘Valencia’, fruit harvested from 4-year-old trees at Eagle Lake, Florida (note that
‘Valencia’ did not pass maturity standards in late February 2024).

| | | | Lbs. | | | | | Lbs. | | |
|---|---|---|---|---|---|---|---|---|---|---|
| | Wt. | Wt. | Juice | | | | | Solids | | Avg. |
| | Sample | Juice | per | % | Total | | Bin | per | Juice | Fruit |
| Sample ID | (Lbs.) | (Lbs.) | Box | Acid | ° Brix | Ratio | No. | Box | Color | Wt. |
| ‘OLL-DC- | 17.98 | 10.36 | 51.858 | 0.97 | 9.54 | 9.84 | 46 | 4.95 | 39.39 | 0.39 |
| 3-40’ Eagle | | | | | | | | | | |
| ‘OLL-DC- | 18.22 | 10.50 | 51.866 | 0.85 | 9.14 | 10.75 | 43 | 4.74 | 39.10 | 0.44 |
| 3-36’ Eagle | | | | | | | | | | |
| ‘OLL-8’ | 21.30 | 11.79 | 49.817 | 0.96 | 9.41 | 9.80 | 51 | 4.69 | 38.87 | 0.42 |
| Eagle | | | | | | | | | | |
| ‘Valencia’ | 20.38 | 11.19 | 49.416 | 1.14 | 8.93 | 7.83 | 63 | 4.41 | 37.33 | 0.32 |
| Eagle | | | | | | | | | | |

1. A new and distinct sweet orange plant named ‘OLL-DC-3-36’ as illustrated and described herein.
